(19) United States
(12) Patent Application Publication
(10) Pub. No.: US 2024/0177802 A1
(43) Pub. Date: May 30, 2024

(54) ACCURATELY PREDICTING VARIANTS FROM METHYLATION SEQUENCING DATA

(71) Applicant: Illumina, Inc., San Diego, CA (US)

(72) Inventors: Daniel Andrews, Cambridge (GB); James Baye, Cambridge (GB)

(21) Appl. No.: 18/523,485

(22) Filed: Nov. 29, 2023

Related U.S. Application Data
(60) Provisional application No. 63/385,593, filed on Nov. 30, 2022.

(51) Int. Cl. G16B 20/20

(52) U.S. Cl. CPC G16B 20/20

(57) ABSTRACT

This disclosure describes methods, non-transitory computer readable media, and systems that can utilize methylation sequencing assay data to generate genotype calls efficiently and accurately for a target genomic sample. In some implementations, the disclosed system identifies the target genomic sample's nucleotide reads comprising nucleobases converted by a methylation sequencing assay. The disclosed system can determine variant calls based on aligning the nucleotide reads with a reference genome. To account for errors introduced by the methylation sequencing assay, in some cases, the disclosed system corrects or modifies genotype-likelihood metrics for a subset of candidate variant calls. The disclosed system further imputes genotype calls based on such modified genotype-likelihood metrics and a comparison of a subset of variant calls with marker variants from a reference panel.

### PRIORITY APPLICATION

The present application claims the benefit of, and priority to, U.S. Provisional Application No. 63/385,593, titled, “ACCURATELY PREDICTING VARIANTS FROM METHYLATION SEQUENCING DATA,” filed Nov. 30, 2022. The aforementioned application is hereby incorporated by reference in its entirety.

### BACKGROUND

In recent years, biotechnology firms and research institutions have improved hardware and software for both determining nucleobase calls and methylation status for genomic samples. For instance, some existing sequencing machines and sequencing-data-analysis software (together “existing sequencing systems”) determine individual nucleobases within sequences by using conventional Sanger sequencing or sequencing-by-synthesis (SBS) methods. When using SBS, existing sequencing systems can monitor many millions of oligonucleotides being synthesized in parallel from templates to predict nucleobase calls for growing nucleotide reads. For instance, a camera in many existing sequencing systems captures images of irradiated fluorescent tags incorporated into oligonucleotides. After capturing such images, some existing sequencing systems process the image data from the camera and determine nucleobase calls for nucleotide reads corresponding to the oligonucleotides. Based on a comparison of the nucleobase calls for such reads and a reference genome, existing systems utilize a variant caller to identify variants in a genomic sample, such as single nucleotide polymorphisms (SNPs), insertions or deletions (indels), or other variants within the genomic sample.

As mentioned, biotechnology firms and research institutions have also improved methods of detecting methylation of cytosine bases at particular genomic regions (e.g., regions encoding or promoting genes) and detecting methylation of larger nucleotide fragments or whole genomes of a sample. For instance, some existing sequencing systems can use sequencing devices and corresponding sequencing-data-analysis software to identify when a methyl or hydroxymethyl group has been added to a cytosine base of a sample's deoxyribonucleic acid (DNA)—where the methylated cytosine base is often part of a cytosine-guanine-dinucleotide pair in a 5′-C-phosphate-G-3′ (CpG) configuration in mammals. For example, existing sequencing systems can detect methylated cytosines by (i) enzymatically converting methylated or unmethylated cytosine bases at CpG or other sites from a sample nucleotide fragment into uracil bases (e.g., dihydrouracil); (ii) determining base calls of nucleotide reads for the sample using a sequencing device, where the sequencing device detects the uracil bases as thymine bases during polymerase chain reaction (PCR) amplification; and (iii) comparing the base calls from the nucleotide reads to a reference genome or non-enzymatically converted nucleotide reads from the sample. Based on the comparison of nucleotide reads from the sample to a reference genome or the non-enzymatically converted nucleotide reads, existing sequencing systems can identify thymine bases from the nucleotide reads that do not match cytosine bases at CpG or other sites within the reference genome or the non-enzymatically converted nucleotide reads and thereby detect methylated cytosine bases in a sample nucleotide fragment.

Despite these recent advances, existing sequencing and methylation detection systems face several shortcomings. For example, existing systems frequently determine inaccurate variant calls from methylation data. In particular, existing systems that attempt to generate variant calls from methylation data often inaccurately determine base calls for genomic regions comprising converted methylated or unmethylated cytosine bases. Such cytosine conversion introduces noise into methylation sequencing data that hinders accurate variant calling. Some existing systems attempt to improve the accuracy of such methylation-data-based variant calling by correcting data, ignoring data, or creating statistical models that account for difficult-to-call genomic regions. However, existing approaches yield limited benefits in accuracy. To illustrate, state-of-the-art sequencing systems call SNPs from methylation data with approximately 0.95 precision and recall. Thus, even the state-of-the-art calls variants from methylation sequencing data with an accuracy demonstrating room for improvement.

In addition to accuracy challenges, some existing sequencing and methylation detection systems are inefficient and consume an inordinate amount of processing time on specialized sequencing devices. Because existing sequencing systems often fail to accurately sequence genomic samples using methylation data, existing systems often perform genomic sequencing separately from a methylation assay. Accordingly, some existing systems require multiple samples from a single organism on which to perform both sequencing and methylation assays in separate computational analyses. To illustrate, existing systems often require the input of a genomic sample for nucleobase sequencing and a separate genomic sample for methylation detection. The duplication of genomic samples often necessitates a duplication of computer processing, computer storage, software programs, and other resources to sequence and determine methylation levels for the same genomic sequence. Thus, existing systems often consume excessive genomic samples, significant time, and computer processing resources to both sequence and determine methylation data for a single genomic sequence.

These, along with additional problems and issues exist in existing sequencing and methylation determination systems.

### SUMMARY

This disclosure describes one or more embodiments of systems, methods, and non-transitory computer readable storage media that solve one or more of the problems described above or provide other advantages over the art. In particular, the disclosed system accurately and efficiently determines variant calls from methylation sequencing data. The disclosed system improves the accuracy of variant calling by imputing, from a variant reference panel, variant calls for genotype calls corresponding to cytosine conversion. For instance, in some embodiments, the disclosed system improves existing SNP or other variant calling by (i) reducing a value of genotype likelihoods for variant calls corresponding to nucleobases converted by a methylation sequencing assay and (ii) imputing SNP calls or other variant calls using a reference panel and the reduced genotype likelihoods.

To illustrate, in one embodiment, the disclosed system identifies, for a target genomic sample, nucleotide reads comprising one or more nucleobases converted by a methylation sequencing assay. The disclosed system may further determine variant calls for the target genomic sample based on an alignment of the nucleotide reads with a reference genome or non-enzymatically converted nucleotide reads. In some implementations, the disclosed system accesses a reference panel comprising marker variants for different haplotypes corresponding to a target genomic region of the target genomic sample and imputes one or more genotype calls for the target genomic sample based on a comparison of a subset of variant calls for the target genomic sample and the marker variants from the reference panel. For instance, the disclosed system can reduce genotype likelihoods for thymine variant calls corresponding to a reference cytosine base or adenine variant calls corresponding to a reference guanine base and impute genotype calls for such genomic coordinates based on the reduced genotype likelihoods.

### BRIEF DESCRIPTION OF THE DRAWINGS

The detailed description refers to the drawings briefly described below.

FIG. 1 illustrates a computing-system environment in which a methylation-genotype-imputation system can operate in accordance with one or more embodiments of the present disclosure.

FIGS. 2A-2B illustrate a schematic diagram of the methylation-genotype-imputation system utilizing methylation sequencing assay data to generate variant calls and impute genotype calls in accordance with one or more embodiments of the present disclosure.

FIG. 3 illustrates a schematic diagram of the methylation-genotype-imputation system determining methylation-level values in accordance with one or more embodiments of the present disclosure.

FIG. 4 illustrates the methylation-genotype-imputation system modifying values of genotype likelihood metrics for a subset of candidate variant calls in accordance with one or more embodiments of the present disclosure.

FIG. 5 illustrates the methylation-genotype-imputation system utilizing a reference panel to generate posterior genotype likelihoods as part of imputation in accordance with one or more embodiments of the present disclosure.

FIGS. 6A and 6B illustrate graphs demonstrating variant calling precision and variant calling recall corresponding with various methylation sequencing assay and variant caller combinations in accordance with one or more embodiments of the present disclosure.

FIG. 7 illustrates a graph demonstrating improvements by the methylation-genotype-imputation system to variant calling precision and variant calling recall from imputation in accordance with one or more embodiments of the present disclosure.

FIG. 8 illustrates a flowchart of a series of acts for imputing one or more genotype calls using methylation data in accordance with one or more embodiments of the present disclosure.

FIG. 9 illustrates a block diagram of an example computing device in accordance with one or more embodiments of the present disclosure.

### DETAILED DESCRIPTION

This disclosure describes one or more embodiments of a methylation-genotype-imputation system that utilizes methylation data to accurately determine variant calls using imputation. For instance, the methylation-genotype-imputation system identifies nucleotide reads for a target genomic sample comprising nucleobases converted by a methylation sequencing assay. The methylation-genotype-imputation system may further determine variant calls for the target genomic sample. To improve the accuracy of the variant calls, the methylation-genotype-imputation system can access a reference panel and impute one or more genotypes for target regions within the target genomic sample. For instance, in some embodiments, the methylation-genotype-imputation system (i) reduces a value of genotype likelihoods (e.g., by a percentage) for variant calls corresponding to nucleobases converted or otherwise affected by a methylation sequencing assay and (ii) imputes SNP calls or other variant calls using a reference panel and the reduced genotype likelihoods.

To illustrate, in some embodiments, the methylation-genotype-imputation system identifies, for a target genomic sample, nucleotide reads comprising one or more nucleobases converted by a methylation sequencing assay. The methylation-genotype-imputation system may further determine variant calls for the target genomic sample based on an alignment of the nucleotide reads with a reference genome. Additionally, the methylation-genotype-imputation system accesses a reference panel comprising marker variants for different haplotypes corresponding to a target genomic region of the target genomic sample and imputes one or more genotype calls for the target genomic sample based on a comparison of a subset of variant calls for the target genomic sample and the marker variants form the reference panel.

The methylation-genotype-imputation system identifies nucleotide reads comprising one or more nucleobases converted by a methylation sequencing assay. Generally, different types of methylation assays detect methylated cytosines by converting methylated or unmethylated cytosine bases into uracil bases and subsequently, in some cases, into thymine bases. Subsequently, as oligonucleotides extracted from the genomic sample is duplicated as part of the methylation sequencing assay, complementary strands reflect regions of cytosine-to-thymine substitutions by having adenines in place of guanines. While these conversions aid in the detection of methylation, the conversions may also negatively affect performance and accuracy of variant callers.

At genomic coordinates corresponding to cytosine conversions and other genomic coordinates generally, the methylation-genotype-imputation system determines variant calls for the target genomic sample based on an alignment of the nucleotide reads with a reference genome or non-enzymatically converted nucleotide reads. Generally, the methylation-genotype-imputation system aligns nucleotide reads with a reference genome and determines genetic variants based on nucleobase calls from the aligned nucleotide reads differing from the reference genome. In some implementations, the methylation-genotype-imputation system generates a variant call file (VCF) comprising variant calls for the target genomic sample and genotype-likelihood metrics that indicate likelihoods that a genomic region comprises a particular genotype.

Having generated variant calls, in some implementations, the methylation-genotype-imputation system improves the accuracy of variant calls by modifying values of a subset of genotype-likelihood metrics. As indicated above, the conversions made during the methylation sequencing assay lowers the accuracy of variant callers. To counteract the negative impact of conversions from the methylation sequencing assay, the methylation-genotype-imputation system identifies a subset of candidate variant calls comprising nucleobases converted or otherwise affected by the methylation sequencing assay.

For example, the methylation-genotype-imputation system may compare the variant calls with the reference genome and/or the original genomic sample (e.g., non-enzymatically converted nucleotide reads). The methylation-genotype-imputation system further reduces the values of the subset of genotype-likelihood metrics corresponding to the subset of candidate variant calls. In one example, the methylation-genotype-imputation system reduces values corresponding with all cytosine-to-thymine and guanine-to-adenine conversions. For instance, the disclosed system can reduce prior genotype likelihoods for thymine variant calls differing from a reference cytosine base or adenine variant calls differing from a reference guanine base (e.g., reducing by 80% PHRED-scaled-genotype-likelihood (PL) metrics for the C>T and G>A variant calls) and impute genotype calls for corresponding genomic coordinates based on the reduced genotype likelihoods.

As previously mentioned, the methylation-genotype-imputation system further improves the accuracy of variant calls by utilizing a modified approach to imputation. In particular, the methylation-genotype-imputation system accesses a reference panel comprising marker variants for different haplotypes corresponding to a target genomic region of the target genomic sample. Such a reference panel includes genomic samples from various populations, ancestries, continents, and/or countries. The haplotypes in the reference panel include one or more marker variants, such as single nucleotide polymorphisms (SNPs) or small insertions and/or deletions.

In some implementations, the methylation-genotype-imputation system utilizes the reference panel to impute one or more genotype calls for a target variant of the target genomic sample. To perform such genotype imputation, in some cases, the methylation-genotype-imputation system imputes one or more genotype calls for the target genomic sample based on a comparison of the subset of variant calls for the target genomic sample and the marker variants from the reference panel. To illustrate, in one or more embodiments, the methylation-genotype-imputation system utilizes a genotype imputation model (e.g., Genotype Likelihoods Imputation and PhaSing mEthod (GLIMPSE)) to compare haplotypes represented by the reference panel to the nucleotide reads corresponding to the target genomic sample. By inputting modified values for genotype-likelihood metrics corresponding to converted nucleobases (e.g., C>T and G>A variant calls) into the genotype imputation model and comparing the reference panel's marker variants and phased nucleotide reads of the genomic sample, the methylation-genotype-imputation system generates posterior genotype likelihoods. The posterior genotype likelihoods indicate likelihoods that genomic coordinates or regions of the target genomic sample and/or additional genomic samples exhibit particular genotypes (e.g., A, T, C, or G).

As suggested above, the methylation-genotype-imputation system provides several technical advantages and benefits over existing sequencing systems and methods. For example, the methylation-genotype-imputation system improves accuracy with which sequencing systems determine genotype calls for target variants based on nucleotide reads subject to a methylation sequencing assay. By reducing or otherwise modifying values of a subset of genotype-likelihood metrics for a subset of candidate variant calls, the methylation-genotype-imputation system approximately accounts for errors introduced by the methylation sequencing assay when converting cytosine bases within the nucleotide reads. Furthermore, by utilizing the reference panel to impute genotype calls for a target variant based on modified genotype-likelihood metrics, the methylation-genotype-imputation system improves the accuracy of predicted genotypes in comparison to existing sequencing systems that analyze methylation sequencing data. In contrast to state-of-the-art methods that determine genotype calls from methylation sequencing data with up to 0.95 precision and recall, as described further below, the methylation-genotype-imputation system provides best-in-class performance in calling variants from short-read methylation data with 0.97 recall and 0.997 precision.

In certain embodiments, the methylation-genotype-imputation system accomplishes such precision and recall using a unique combination of a variant call model and a genotype imputation model—by using EpiDiverse and Illumina, Inc.'s DRAGEN Variant Caller (VC) together as a variant call model and GLIMPSE as a genotype imputation model. As demonstrated further below, this combination outperforms other tested combinations. In some cases, accordingly, the methylation-genotype-imputation system imputes a genotype call that differs from—and is more accurate than—an initial variant call by a variant call model (e.g., EpiDiverse+DRAGEN VC or each by itself) at a genomic coordinate for either a C>T variant or a G>A variant.

In part due to the improved genotype-calling accuracy, the methylation-genotype-imputation system improves efficiency in processing and physical resources relative to existing sequencing systems. As noted above, because state-of-the-art genotype-calling accuracy from methylation sequencing data can be unfit for clinical benchmarks, some existing sequencing systems execute (i) a separate methylation sequencing assay to enzymatically convert nucleotide reads from a genomic sample and determine methylation levels and (ii) a separate DNA sequencing run with non-enzymatically converted nucleotide reads from the genomic sample to determine variant calls. Such separate methylation sequencing assays and DNA sequencing can consume and duplicate computer processing, memory storage, physical space and reagents for a nucleotide-sample slide (e.g., flow cell), and software programs (e.g., separate methylation analysis and variant calling software). In contrast to such a bifurcated approach, in some embodiments, the methylation-genotype-imputation system can not only determine methylation-level values indicating levels of methylation of a target genomic sample's cytosine bases, but also utilize methylation assay data to generate variant calls for the genomic sample with improved accuracy. Thus, the methylation-genotype-imputation system can efficiently generate epigenetic and genetic sequencing data from a single genomic sample. By generating both methylation-level values and variant calls from the same genomic sample, the methylation-genotype-imputation system further reduces the amount of computer processing, computer storage, software programs, space used on a nucleotide-sample slide in a sequencing device, and other resources to generate accurate sequencing and methylation data.

As illustrated by the foregoing discussion, the present disclosure utilizes a variety of terms to describe features and advantages of the methylation-genotype-imputation system. As used herein, for example, the term “methylation sequencing assay” refers to an assay that detects, measures, or quantifies methylation of cytosine from an oligonucleotide or other nucleotide sequence. In some cases, a methylation sequencing assay detects or quantifies methylation of cytosine at particular target genomic regions or in particular cell types. As suggested above and explained below, some methylation sequencing assays quantify methylation in terms of methylation-level values.

Relatedly, the term “methylation-level value” refers to a numeric value indicating an amount, percentage, ratio, or quantity of cytosine to which a methyl group or hydroxymethyl group has been added or bonded. For instance, a methylation-level value includes a score (e.g., ranging from 0 to 1) that indicates a percentage or ratio of cytosine bases (e.g., at CpG or other cytosine sites) for particular genomic coordinates or genomic regions to which a methyl group has been added. In some cases, a methylation-level value is expressed as a beta value or an M value. To illustrate, a beta value may estimate a methylation level using a ratio of signal intensities between methylated alleles corresponding to a genomic coordinate and unmethylated alleles corresponding to the genomic coordinate, where 0 represents completely unmethylated and 1 represents completely methylated. By contrast, an M value may represent a log 2 ratio of signal intensities of a methylated probe and an unmethylated probe corresponding to a cytosine base.

As used herein, the term “target genomic sample” refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As used herein, the term “nucleotide read” (or simply “read”) refers to an inferred sequence of one or more nucleobases (or nucleobase pairs) from all or part of a sample nucleotide sequence (e.g., a sample genomic sequence, complementary DNA). In particular, a nucleotide read includes a determined or predicted sequence of nucleobase calls for a nucleotide sequence (or group of monoclonal nucleotide sequences) from a sample library fragment corresponding to a genomic sample. For example, in some cases, a sequencing device determines a nucleotide read by generating nucleobase calls for nucleobases passed through a nanopore of a nucleotide-sample slide, determined via fluorescent tagging, or determined from a cluster in a flow cell.

As further used herein, the term “genotype call” refers to a determination or prediction of a particular genotype of a genomic sample at a genomic locus. In particular, a genotype call can include a prediction of a particular genotype of a genomic sample with respect to a reference genome or a reference sequence at a genomic coordinate or a genomic region. For instance, in some cases, a genotype call includes a determination or prediction that a genomic sample comprises both a nucleobase and a complementary nucleobase at a genomic coordinate that is either homozygous or heterozygous for a reference base or a variant (e.g., homozygous reference bases represented as 0|0 or heterozygous for a variant on a particular strand represented as 0|1). A genotype call is often determined for a genomic coordinate or genomic region at which an SNP, insertion, deletion, or other variant has been identified for a population of organisms.

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or another base-call-output file—based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or a uracil (U) call.

As used herein, the term “nucleobase” refers to a nitrogenous base. In particular, nucleobases comprise components of nucleotides. For example, a nucleobase may be an adenine (A), cytosine (C), guanine (G), or thymine (T).

As used herein, the term “variant call” refers to one or more nucleobase calls that differ from a reference genome or reference sequence at a particular genomic coordinate or genomic region. In particular, a variant call can include a nucleobase call (e.g., SNP) at a genomic coordinate in a genomic sample having a predicted variation from the reference base in the reference genome. Similarly, a variant call can include multiple nucleobase calls (e.g., inversion, indel spanning multiple genomic coordinates) at a genomic region in a genomic sample that differ from the reference bases in the reference genome. Accordingly, a variant call may include, but is not limited to, a single nucleotide variant (SNV), an insertion or a deletion (indel), or a base call that is part of a structural variant.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. GRCh38 may include alternate contiguous sequences representing alternate haplotypes, such as SNPs and small indels (e.g., 10 or fewer base pairs, 50 or fewer base pairs).

As used herein, the term “reference panel” refers to a digital collection or database of haplotypes from genomic samples for which one or more ancestral or progenitorial haplotypes have been determined. In some cases, a reference panel includes a digital database of haplotypes from genomic samples representative of (or common among) an organism's population and for which multiple ancestral or progenitorial haplotypes have been determined. A reference panel can likewise include a data file or other organization of data reflecting genomic sequences and various variant markers (e.g., SNPs) in those genomic sequences. To illustrate, a reference panel can include data corresponding to genomic sequences and various tags or other metadata characterizing or categorizing the genomic sequences. In some cases, the methylation-genotype-imputation system accesses an initial reference panel developed by the Haplotype Reference Consortium (HRM), 1000 Genomes Project, or Illumina, Inc. when generating a reference panel comprising marker-variant indicators for marker variants at genomic coordinates corresponding to genomic samples of different haplotypes.

As used herein, the term “marker variant” refers to a variant at a polymorphic site in a population. In particular, a marker variant includes one of two or more alleles present among a population at a polymorphic genomic coordinate or genomic region at a frequency greater than a threshold frequency, such as greater than 1% of a population. In some cases, a marker variant includes SNPs present at a polymorphic genomic coordinate among a human population that is represented in a reference panel. Additionally, or alternatively, a marker variant can include insertions or deletions (indels), structural variants, or other variants at polymorphic sites among a population. As suggested above, alleles for particular haplotypes represented by a reference panel may include SNPs or other variant markers used for imputation.

As used herein, the term “haplotype” refers to nucleotide sequences that are present in an organism (or present in organisms from a population) and inherited from one or more ancestors. In particular, a haplotype can include alleles or other nucleotide sequences present in organisms of a population and inherited together by such organisms respectively from a single parent. In one or more embodiments, haplotypes include a set of SNPs on the same chromosome that tend to be inherited together. In some cases, data representing a haplotype or a set of different haplotypes are stored or otherwise accessible on a haplotype database.

Additionally, as used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-COV-2 for a reference genome for the SARS-CoV-2 virus) and a position of a nucleotide-base within the source for the reference genome (e.g., mt:16568 or SARS-COV-2:29001). By contrast, in certain cases, a genomic coordinate refers to a position of a nucleotide-base within a reference genome without reference to a chromosome or source (e.g., 29727).

As used herein, the term “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870). Relatedly, the term “target genomic region” refers to a particular genomic region targeted for imputation. In particular, a target genomic region refers to a range of genomic coordinates at which imputation is desirable. For example, a target genomic region may comprise a genomic region at which nucleobase calls correspond to a cytosine base that has been converted to a uracil or thymine base by a methylation sequencing assay, a complementary guanine base that was change or converted as a result of the cytosine-base conversion, and/or variant calls are missing or are associated with confidence scores below a threshold value.

As used herein, the term “genotype-likelihood metric” refers to a value indicating a likelihood, probability, or score of a particular genotype at a genomic coordinate or genomic region. For instance, a genotype-likelihood metric includes a value indicating a likelihood of a homozygous reference genotype, a likelihood of a heterozygous variant genotype, or a likelihood of a homozygous variant genotype at one or more genomic coordinates. A genotype likelihood can include a specialized prediction depending on the application of the methylation-genotype-imputation system, such as for predicting SNPs. In some cases, a genotype-likelihood metric may comprise a PHRED-scaled-genotype-likelihood metric.

Relatedly, the term “genotype imputation model” refers to an algorithm or model for imputing genotypes of genomic regions based on sequencing data from a genomic sample and haplotypes corresponding to respective genomic regions. In particular, a genotype imputation model includes a hidden Markov model (HMM)-based algorithm or model for imputing genotypes of genomic regions and phasing haplotypes based on sequencing data from a genomic sample and haplotypes corresponding to respective genomic regions from a haplotype reference panel. As indicated above, in some cases, a genotype imputation model includes GLIMPSE. Alternatively, a genotype imputation models includes fastPHASE, BEAGLE, MACH, or IMPUTE.

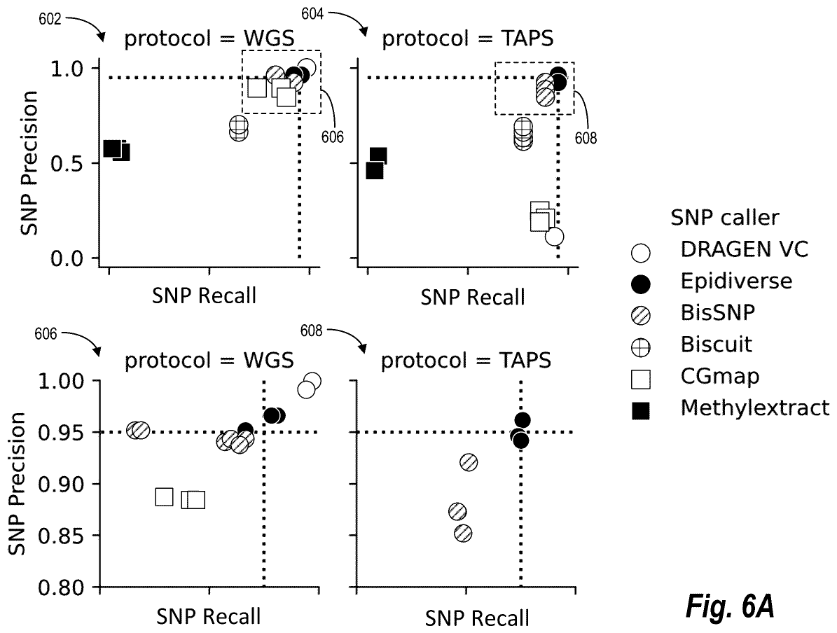

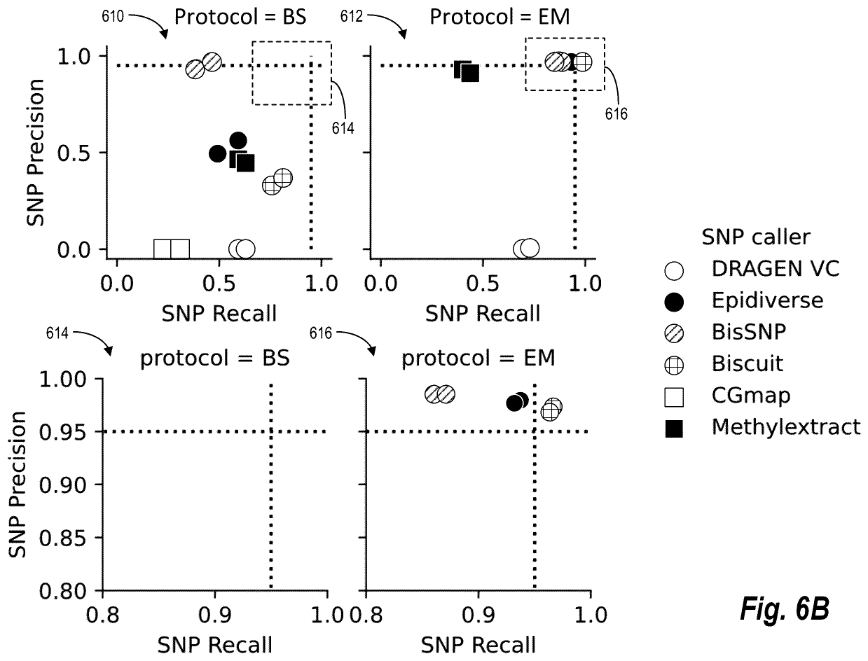

As used herein, the term “variant call model” (or simply (“variant caller”) refers to a probabilistic model that generates rapid sequencing data from nucleotide reads of a sample nucleotide sequence, including variant calls and associated metrics. For example, in some cases, a variant call model refers to a Bayesian probability model that generates variant calls based on nucleotide reads of a sample nucleotide sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A variant call model may likewise include multiple components, including, but not limited to, different software applications or components for mapping and aligning, sorting, duplicate marking, computing read pileup depths, and variant calling. In some cases, the variant call model refers to the ILLUMINA DRAGEN model for variant calling functions and mapping and alignment functions. Additional examples are provided below with respect to FIG. 6B.

As mentioned, in some embodiments, the methylation-genotype-imputation system modifies genotype-likelihood metrics other metrics in data fields of a variant call file. As used herein, the term “variant call file” refers to a digital file that indicates or represents one or more nucleotide base calls, genotype calls, and/or variant calls compared to a reference genome along with other information pertaining to the calls. For example, a variant call format (VCF) file refers to a text file format that contains information about variants at specific genomic coordinates, including meta-information lines, a header line, and data lines where each data line contains information about a single nucleobase call (e.g., a single variant). In some cases, the methylation-genotype-imputation system can generate different versions of variant call files, including a pre-filter variant call file comprising variant calls that either pass or fail a quality filter for base-call-quality metrics or a post-filter variant call file comprising variant calls that pass the quality filter but excludes variant calls that fail the quality filter.

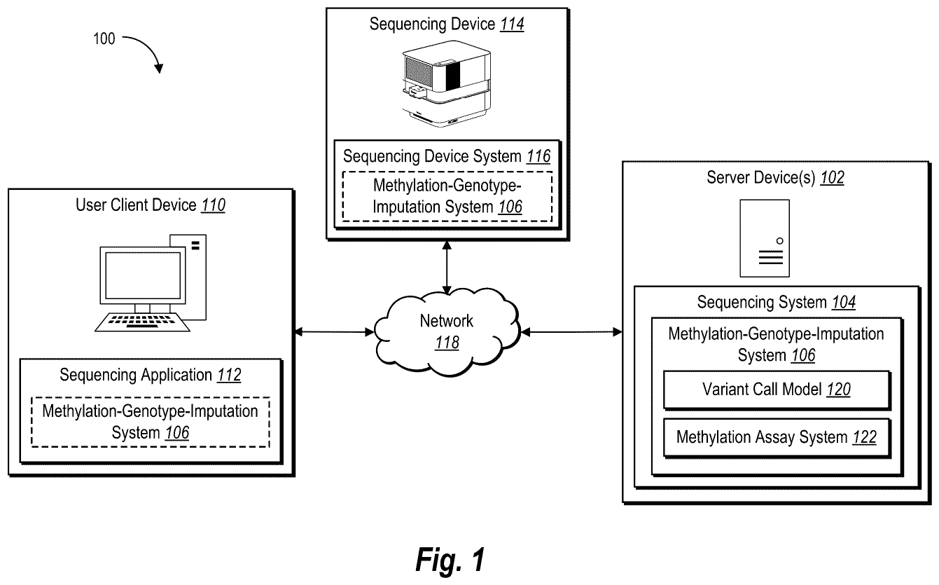

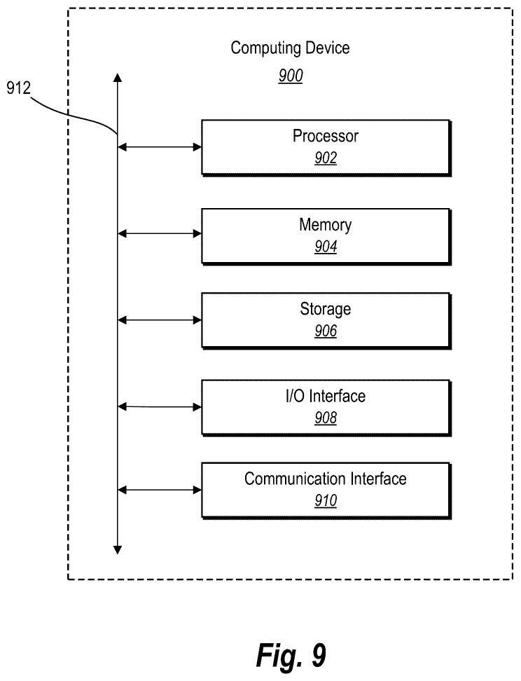

The following paragraphs describe the methylation-genotype-imputation system with respect to illustrative figures that portray example embodiments and implementations. For example, FIG. 1 illustrates a schematic diagram of a computing system 100 in which a methylation-genotype-imputation system 106 operates in accordance with one or more embodiments. As illustrated, the computing system 100 includes server device(s) 102, a sequencing device 114, and a user client device 110 connected via a network 118. While FIG. 1 shows an embodiment of the methylation-genotype-imputation system 106, this disclosure describes alternative embodiments and configurations below. As shown in FIG. 1, the sequencing device 114, the server device(s) 102, and the user client device 110 can communicate with each other via the network 118. The network 118 comprises any suitable network over which computing devices can communicate. Example networks are discussed in additional detail below with respect to FIG. 9.

As indicated by FIG. 1, the sequencing device 114 comprises a sequencing device system 116 for sequencing a genomic sample or other nucleic-acid polymer, such as when sequencing oligonucleotides extracted from a genomic sample as part of a methylation sequencing assay. In some embodiments, by executing the sequencing device system 116, the sequencing device 114 analyzes nucleotide sequences or oligonucleotides extracted from genomic samples to generate nucleotide reads or other data utilizing computer implemented methods and systems (described herein) either directly or indirectly on the sequencing device 114. More particularly, the sequencing device 114 receives nucleotide-sample slides (e.g., flow cells) comprising nucleotide sequences extracted from samples and then copies and determines the nucleobase sequence of such extracted nucleotide sequences. As part of a methylation sequencing assay, for instance, the sequencing device 114 may determine nucleobase calls for nucleotide reads comprising CpG sites or other cytosine sites.

As suggested above, by executing the sequencing device system 116, the sequencing device 114 can run one or more sequencing cycles as part of a sequencing run. By executing the methylation-genotype-imputation system 106, for instance, the sequencing device 114 can (i) sequence certain uracil bases that were converted from methylated cytosine bases and that are part of a nucleotide read and (ii) determine nucleobase calls of thymine for such uracil bases as part of a methylation sequencing assay. In one or more embodiments, the sequencing device 114 utilizes Sequencing by Synthesis (SBS) to sequence nucleic-acid polymers into nucleotide reads.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run a sequencing system 104 and/or the methylation-genotype-imputation system 106 to generate, receive, analyze, store, and transmit digital data, such as by receiving base-call data, methylation assay data, or determining variant calls based on analyzing such base-call data and/or methylation assay data.

As further suggested by FIG. 1, the sequencing device 114 may send (and the server device(s) 102 may receive) base-call data generated during a sequencing run of the sequencing device 114. By executing software in the form of the sequencing system 104 or the methylation-genotype-imputation system 106, the server device(s) 102 may align nucleotide reads with a reference genome and determine variant calls based on the aligned nucleotide reads. The server device(s) 102 may also communicate with the user client device 110. In particular, the server device(s) 102 can send data to the user client device 110, including a variant call file (VCF), or other information indicating nucleobase calls, sequencing metrics, error data, or other metrics.

In some embodiments, the server device(s) 102 comprise a distributed collection of servers where the server device(s) 102 include a number of server devices distributed across the network 118 and located in the same or different physical locations. Further, the server device(s) 102 can comprise a content server, an application server, a communication server, a web-hosting server, or another type of server.

As further illustrated and indicated in FIG. 1, the user client device 110 can generate, store, receive, and send digital data. In particular, the user client device 110 can receive variant calls and corresponding sequencing metrics from the server device(s) 102 or receive base-call data (e.g., BCL or FASTQ) and corresponding sequencing metrics from the sequencing device 114.

Furthermore, the user client device 110 may communicate with the server device(s) 102 to receive a VCF comprising nucleobase calls and/or other metrics, such as base-call-quality metrics or pass-filter metrics. The user client device 110 can accordingly present or display information pertaining to variant calls or other nucleobase calls within a graphical user interface to a user associated with the user client device 110. In particular, the user client device 110 can present results from a methylation sequencing assay or graphics that indicate either or both of methylation-level values and corrected methylation-level values for target cytosine bases.

Although FIG. 1 depicts the user client device 110 as a desktop or laptop computer, the user client device 110 may comprise various types of client devices. For example, in some embodiments, the user client device 110 includes non-mobile devices, such as desktop computers or servers, or other types of client devices. In yet other embodiments, the user client device 110 includes mobile devices, such as laptops, tablets, mobile telephones, or smartphones. Additional details regarding the user client device 110 are discussed below with respect to FIG. 9.

As further illustrated in FIG. 1, the user client device 110 includes a sequencing application 112. The sequencing application 112 may be a web application or a native application stored and executed on the user client device 110 (e.g., a mobile application, desktop application). The sequencing application 112 can include instructions that (when executed) cause the user client device 110 to receive data from the methylation-genotype-imputation system 106 and present, for display at the user client device 110, base-call data (e.g., from a BCL), data from a VCF, or data from a methylation sequencing assay.

As further illustrated in FIG. 1, a version of the methylation-genotype-imputation system 106 may be located on the user client device 110 as part of the sequencing application 112 or on the sequencing device 114 as part of the sequencing device system 116. In some embodiments, the methylation-genotype-imputation system 106 is implemented by (e.g., located entirely or in part) on the user client device 110. In yet other embodiments, the methylation-genotype-imputation system 106 is implemented by one or more other components of the computing system 100, such as the sequencing device 114. In particular, the methylation-genotype-imputation system 106 can be implemented in a variety of different ways across the sequencing device 114, the user client device 110, and the server device(s) 102. As illustrated in FIG. 1, the methylation-genotype-imputation system 106 is implemented by (e.g., entirely or in part) the sequencing system 104 implemented by the server device(s) 102. In at least one example, the methylation-genotype-imputation system 106 can be downloaded from the server device(s) 102 to the sequencing device 114 and/or the user client device 110 where all or part of the functionality of the methylation-genotype-imputation system 106 is performed at each respective device within the computing system.

As further illustrated in FIG. 1, the methylation-genotype-imputation system 106 may implement a variant call model 120 and a methylation assay system 122. By executing the variant call model 120, the methylation-genotype-imputation system 106 may align nucleotide reads with a reference genome and determine variant calls based on the aligned nucleotide reads. The methylation assay system 122 is also implemented by the methylation-genotype-imputation system 106. The methylation assay system 122 determines methylation-level values for CpG sites or other cytosine sites. As previously mentioned, the variant call model 120 and/or the methylation assay system 122 may be implemented by the methylation-genotype-imputation system 106.

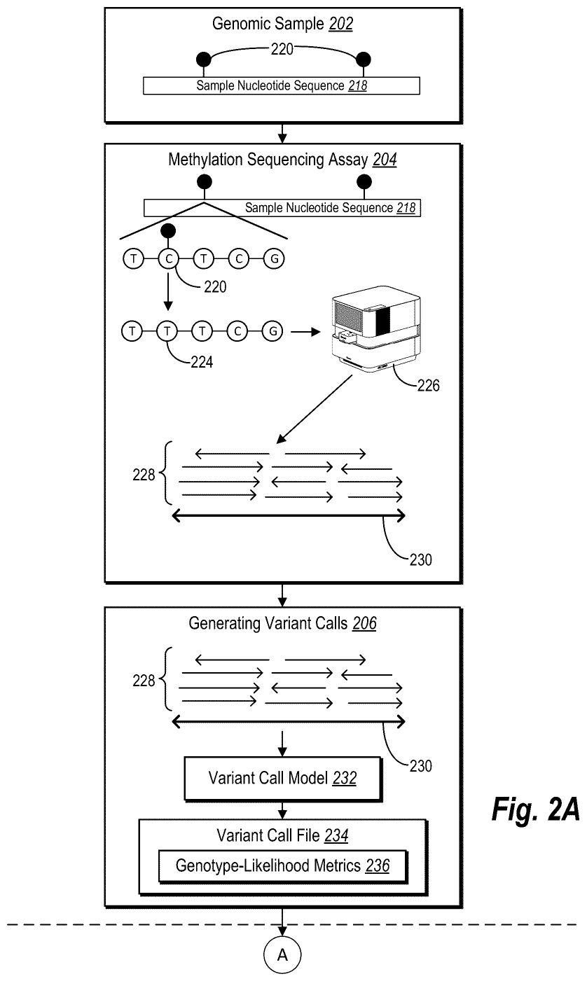

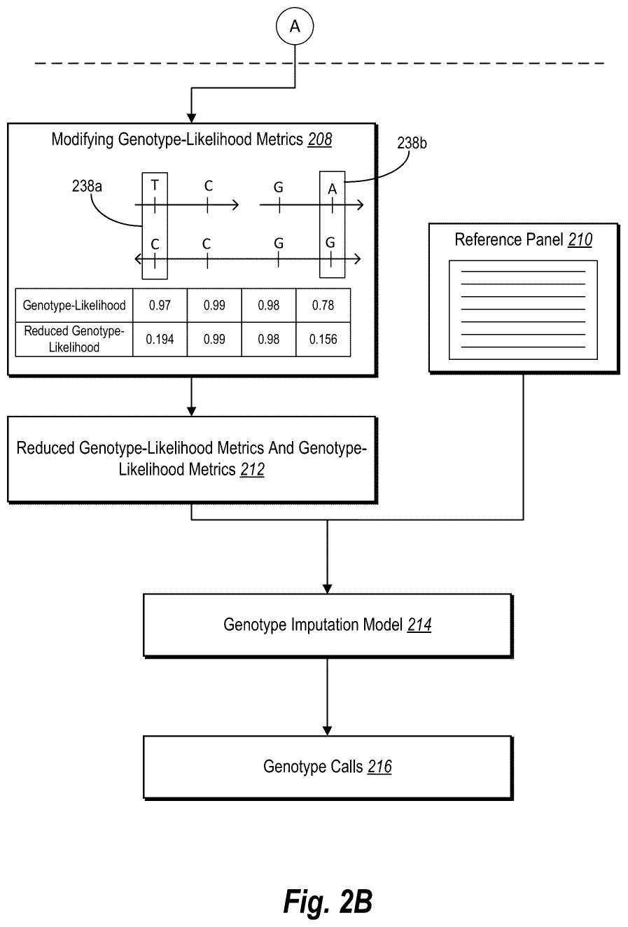

As indicated above, the methylation-genotype-imputation system 106 can accurately and efficiently generate variant calls using methylation sequencing data. In accordance with one or more embodiments, FIGS. 2A-2B illustrate the methylation-genotype-imputation system 106 generating genotype calls using methylation sequencing data in accordance with one or more embodiments. As an overview, as shown in FIGS. 2A-2B, the methylation-genotype-imputation system 106 (i) utilizes a methylation sequencing assay to predict methylated and unmethylated cytosine cites within a target genomic sample, (ii) generates variant calls comprising genotype-likelihood metrics, (iii) modifies the genotype-likelihood metrics to account for inaccuracies introduced by the methylation sequencing assay, and (iv) utilizes a genotype imputation model to generate genotype calls for the genomic sample.

As shown in FIG. 2A, for instance, the methylation-genotype-imputation system 106 identifies methylation-level values for a genomic sample 202. As illustrated, the genomic sample 202 comprises (or has extracted from it) a sample nucleotide sequence 218. In turn, the sample nucleotide sequence 218 comprises methylated cytosine bases 220. In some implementations, the methylation-genotype-imputation system 106 utilizes a methylation sequencing assay 204 to determine methylation-level values for the genomic sample 202. In particular, the methylation-genotype-imputation system 106 identifies methylation-level values for cytosine bases within the genomic sample 202 by either (i) accessing or receiving the methylation-level values from a computing device or (ii) determining the methylation-level values for the cytosine bases using the methylation sequencing assay 204. For example, in some cases, the methylation-genotype-imputation system 106 inputs or runs the genomic sample 202 through the methylation sequencing assay 204.

As illustrated in FIG. 2A, the sample nucleotide sequence 218 comprises one or more cytosine bases. The sample nucleotide sequence 218 comprises both unmethylated cytosine bases and methylated cytosine bases. In certain cases, the sample nucleotide sequence 218 constitutes a sample library fragment with genomic DNA from a sample comprising the methylated cytosine bases 220. Consistent with the disclosure above, in certain implementations, the methylation-genotype-imputation system 106 utilizes an enzyme to convert methylated or unmethylated cytosine bases to uracil bases or thymine bases 224 as part of the methylation sequencing assay 204.

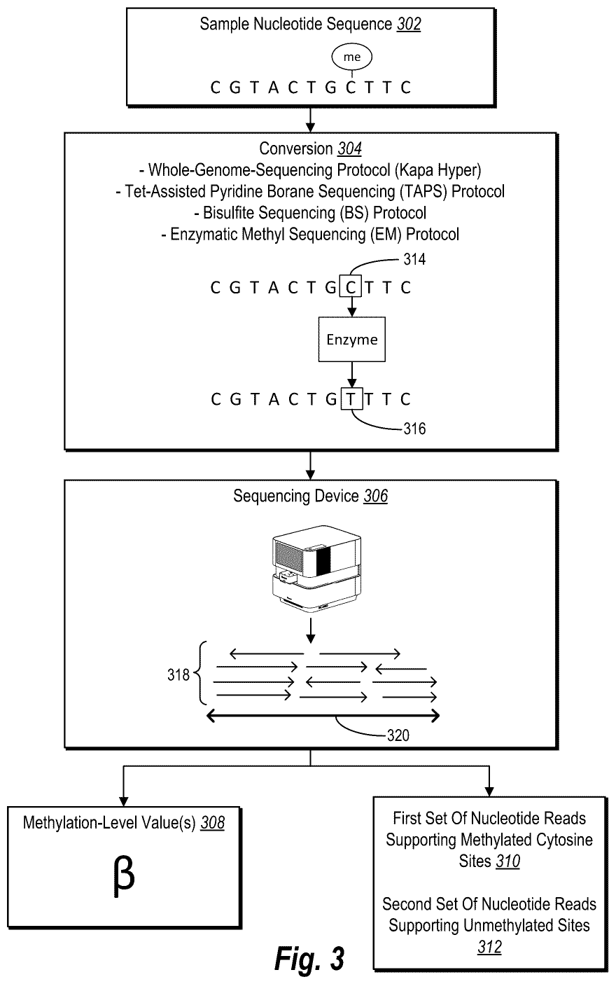

As further part of the methylation sequencing assay 204, in some embodiments, the methylation-genotype-imputation system 106 amplifies and determines nucleobase calls for the sample nucleotide sequence 218 and complementary strands using a sequencing device 226 (e.g., the sequencing device 114 illustrated in FIG. 1). More specifically, the methylation-genotype-imputation system 106 utilizes SBS to determine thymine nucleobase calls for one or more of the cytosine bases that have been converted into uracil bases or thymine bases 224. In some embodiments, the methylation-genotype-imputation system 106 compares the nucleotide reads 228 with a reference genome 230 to identify converted cytosine bases. FIG. 3 and the corresponding paragraphs further detail example methylation sequencing assays utilized by the methylation-genotype-imputation system 106 in one or more embodiments.

As further indicated by FIG. 2A, in certain implementations, the methylation-genotype-imputation system 106 can perform an act 206 of generating variant calls. In some implementations, the methylation-genotype-imputation system 106 uses the server device(s) 102 to align the nucleotide reads 228 with the reference genome 230 to determine variant calls. More specifically, the methylation-genotype-imputation system 106 utilizes a variant call model 232 to generate a variant call file (VCF) 234. Generally, the VCF 234 comprises a base-call-output file that comprises data representing nucleotide reads corresponding to various genomic coordinates. For example, the VCF 234 comprises nucleobase calls, variant calls, and/or corresponding metrics, such as genotype-likelihood metrics 236. The genotype-likelihood metrics 236 generally indicate the likelihood that a genomic region or coordinate comprises a particular genotype. In some implementations, the genotype-likelihood metrics 236 are based on the nucleotide reads 228 from the genomic sample and quality scores for the nucleotide reads and/or other sequencing metrics.

As illustrated in FIG. 2B, the methylation-genotype-imputation system 106 performs an act 208 of modifying genotype-likelihood metrics. In some implementations, the methylation-genotype-imputation system 106 reduces values of a subset of the genotype-likelihood metrics 236 within the VCF 234 to approximately account for errors introduced by the methylation sequencing assay 204. In certain implementations, the methylation-genotype-imputation system 106 identifies nucleotide reads comprising thymine bases or uracil bases converted from cytosine bases by the methylation sequencing assay. Furthermore, the methylation-genotype-imputation system 106 identifies nucleotide reads comprising adenine bases converted from guanine bases during amplification steps in the methylation sequencing assay. As illustrated in FIG. 2B, the methylation-genotype-imputation system 106 identifies nucleotide reads at genomic coordinates 238a-238b as reads comprising converted bases by comparing the nucleotide reads with a reference genome. For example, the methylation-genotype-imputation system 106 determines a thymine base call at the genomic coordinate 238a for which the reference genome comprises a cytosine base. The methylation-genotype-imputation system 106 further determines an adenine base call at the genomic coordinate 238b for which the reference genome comprises a guanine base. In certain implementations, the methylation-genotype-imputation system 106 reduces the genotype-likelihood metrics for the identified subset of genotype-likelihood metrics.

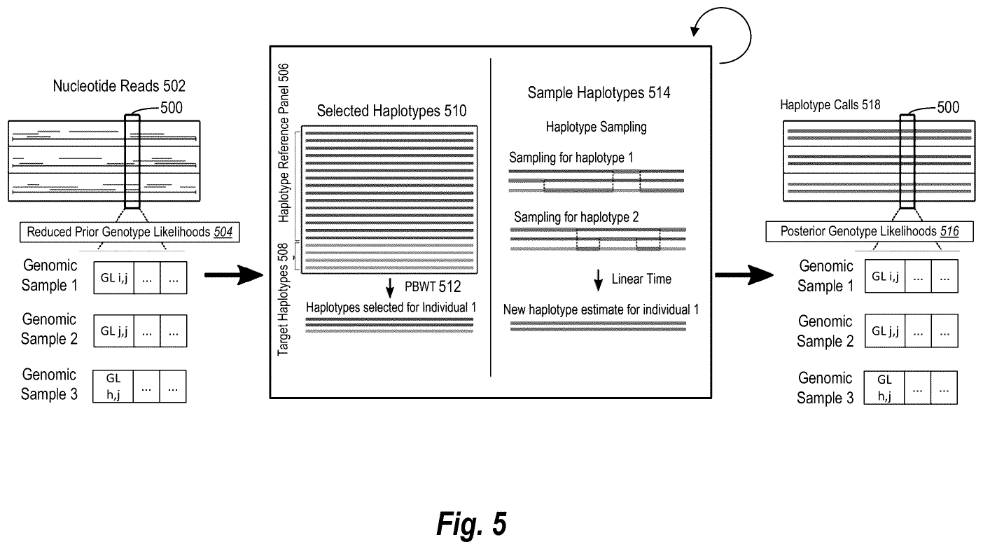

As indicated above, in some implementations, the methylation-genotype-imputation system 106 imputes one or more genotype calls for the genomic sample 202 based on a comparison of the variant calls for the target genomic samples and marker variants from a reference panel 210. As illustrated in FIG. 2B, the methylation-genotype-imputation system 106 accesses the reference panel 210. The reference panel 210 includes a digital representation of haplotypes from various genomic samples, including a variety of quantities of diverse genomic samples. The methylation-genotype-imputation system 106 utilizes a genotype imputation model 214 to analyze reduced genotype-likelihood metrics and genotype-likelihood metrics 212 to generate genotype calls 216. For instance, the methylation-genotype-imputation system 106 can impute genotype calls for genomic coordinates at which (i) thymine variant calls have reduced genotype-likelihood metrics and correspond to a reference cytosine base or (ii) adenine variant calls have reduced genotype-likelihood metrics and correspond to a reference guanine base. FIG. 5 and the corresponding discussion provide additional detail describing how the methylation-genotype-imputation system 106 utilizes the reference panel 210 to generate the genotype calls 216 in accordance with one or more implementations.

As mentioned, the methylation-genotype-imputation system 106 identifies nucleotide reads comprising one or more nucleobases converted by a methylation sequencing assay. FIG. 3 and the corresponding paragraphs further describe various methylation assay protocols and nucleobase conversions in accordance with one or more implementations. By way of overview, the methylation-genotype-imputation system 106 utilizes various methylation sequencing protocols to convert methylated or unmethylated cytosine bases to thymine bases or uracil bases, utilizes a sequencing device to identify converted bases, and generates methylation-level values.

As shown in FIG. 3, for instance, the methylation-genotype-imputation system 106 identifies methylation-level value(s) 308 for cytosine bases within a sample nucleotide sequence 302 determined by a methylation sequencing assay. For example, in some cases, the methylation-genotype-imputation system 106 inputs or runs the sample nucleotide sequence 302 through a methylation sequencing assay, such as a Whole Genome Sequencing (WGS) protocol (e.g., Kapa Hyper), Tet-Assisted Pyridine borane Sequencing (TAPS), Bisulfite Sequencing (BS), Enzymatic Methyl sequencing (EM), or another assay. In certain embodiments, as part of the above-listed methylation sequencing assays, the methylation-genotype-imputation system 106 uses various enzymes to perform a conversion 304 by which methylated or unmethylated cytosine bases 314 are converted into thymine bases 316 or uracil bases. Some methylation sequencing assays, including the TAPS protocol, use enzymes to convert methylated cytosines to uracil. More specifically, in the TAPS protocol, the methylation-genotype-imputation system 106 uses a TET enzyme to convert methylated cytosine bases to uracil bases, which are converted after amplification (e.g., polymerase chain reaction) to thymine bases. Other methylation sequencing assays, such as the BS protocol and the EM sequencing protocol, convert unmethylated cytosine bases to uracil bases. To illustrate, in the BS protocol, bisulfite is used to convert unmethylated cytosine to uracil while 5-methylcytosine residues are unaffected. In another example, as part of the EM sequencing protocol, the methylation-genotype-imputation system 106 uses enzymatic reactions using TET2 and APOBEC3A to convert unmethylated cytosine bases to uracil bases.

As further part of the methylation sequencing assay, the methylation-genotype-imputation system 106 can amplify and determine variant calls for the sample nucleotide sequence 302 and complementary strands using a sequencing device 306. In some such cases, the methylation-genotype-imputation system 106 uses SBS to determine nucleobase calls for the sample nucleotide sequence 302 when sequencing or amplifying a nucleotide read of nucleotide reads 318. In some implementations, the methylation-genotype-imputation system 106 aligns the nucleotide reads 318 with a reference genome 320 to determine variant calls. More specifically, the methylation-genotype-imputation system 106 utilizes the sequencing device 306 to identify uracil bases or thymine bases that have been converted from cytosine bases to determine locations of methylated or unmethylated cytosine bases in the genomic sample 202. As part of the methylation sequencing assay, the methylation-genotype-imputation system 106 identifies thymine or uracil bases in the nucleotide reads 318 that vary from cytosine bases at the same genomic coordinates within the reference genome 320. By contrast, in some embodiments, the methylation-genotype-imputation system 106 compares the nucleotide reads 318 with non-enzymatically converted nucleotide reads from a same genomic sample and thereby identifies thymine or uracil bases in the nucleotide reads 318 that vary from reference cytosine bases.

Additionally, in some embodiments, the methylation-genotype-imputation system 106 amplifies and determines variant calls for complementary strands of the sample nucleotide sequence 302. To illustrate, during amplification, complementary strands of the sample nucleotide sequence 302 include adenine bases that pair with converted uracil or thymine bases in the sample nucleotide sequence 302. To determine that an adenine base corresponds to a converted uracil or thymine base, the methylation-genotype-imputation system 106 utilizes the sequencing device 306 to sequence complementary nucleotide reads and compares the complementary nucleotide reads with the reference genome 320. By contrast, in some embodiments, the methylation-genotype-imputation system 106 compares the complementary nucleotide reads with non-enzymatically converted nucleotide reads from a same genomic sample.

As further shown in FIG. 3, the methylation-genotype-imputation system 106 determines methylation-level value(s) 308 for the cytosine bases as part of the methylation sequencing assay. For instance, in some cases, the methylation-genotype-imputation system 106 determines beta value(s) that each indicate a percentage or ratio of the nucleotide reads 318 covering cytosine bases to which a methyl group or hydroxymethyl group has been added. In particular, the beta value may estimate a methylation level using a ratio of signal intensities between methylated alleles corresponding to a genomic coordinate for a cytosine base and unmethylated alleles corresponding to the genomic coordinate for the cytosine base. Alternatively, the methylation-level value(s) 308 may each constitute an M value that indicates a log 2 ratio of signal intensities of a methylated probe corresponding to a cytosine base and an unmethylated probe corresponding to the cytosine base.

As further shown in FIG. 3, in some embodiments, the methylation-genotype-imputation system 106 further identifies, from data generated by the methylation sequencing assay, a first set of nucleotide reads supporting methylated cytosine sites 310 within the sample nucleotide sequence 302 (or a genomic sample more generally) and a second set of nucleotide reads supporting unmethylated cytosine sites 312 within the sample nucleotide sequence 302 (or the genomic sample). For instance, the methylation-genotype-imputation system 106 identifies the first set of nucleotide reads supporting methylated cytosine sites 310 and the second set of nucleotide reads supporting unmethylated cytosine sites 312 based on the alignment between the nucleotide reads 318 and the reference genome 320. In some embodiments, the first set of nucleotide reads and the second set of nucleotide reads may be specific to methylated and unmethylated cytosine bases at particular genomic coordinates.

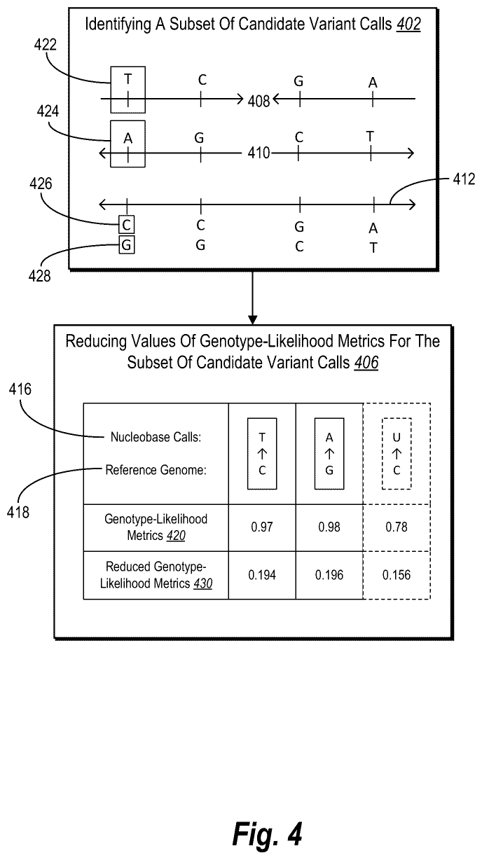

While methylation sequencing assays provide epigenetic information for a genomic sample, methylation sequencing assays also introduce errors that negatively impact the accuracy of variant calling. The methylation-genotype-imputation system 106 modifies data from methylation sequencing assays to generate variant calls more efficiently and accurately than existing sequencing systems. More specifically, in some cases, the methylation-genotype-imputation system 106 modifies genotype-likelihood metrics for an identified subset of candidate variant calls. By way of overview, FIG. 4 illustrates the methylation-genotype-imputation system 106 identifying a subset of candidate variant calls that have likely been affected by the methylation sequencing assay and modifies values of genotype-likelihood metrics for the identified subset of candidate variant calls.

As illustrated in FIG. 4, for instance, the methylation-genotype-imputation system 106 performs an act 402 of identifying a subset of candidate variant calls. Generally, the methylation-genotype-imputation system 106 identifies variant calls that have been influenced by the methylation sequencing assay. In some cases, the methylation-genotype-imputation system 106 identifies nucleotide reads comprising thymine bases or uracil bases converted from cytosine bases by the methylation sequencing assay. For instance, the methylation-genotype-imputation system 106 may align nucleotide reads 408 and corresponding complementary nucleotide reads 410 with a reference genome 412 and identify nucleobases that were likely converted during the methylation sequencing assay. More specifically, in some embodiments, the methylation-genotype-imputation system 106 identifies thymine or adenine base calls that correspond with cytosine or guanine bases in the reference genome 412, respectively.

As an example, and as illustrated in FIG. 4, the methylation-genotype-imputation system 106 determines that a thymine base call 422 in the nucleotide reads 408 aligns with a cytosine base 426 in the reference genome 412. The methylation-genotype-imputation system 106 may determine that the thymine base call 422 was converted from a cytosine base during a methylation sequencing assay. The corresponding complementary nucleotide reads 410 comprise an adenine base call 424 aligning with a guanine base 428 in the reference genome 412. In some implementations, methylation-genotype-imputation system 106 determines that the thymine base call 422 and the adenine base call 424 are within the subset of candidate variant calls corresponding to nucleobases converted from cytosine bases by the methylation sequencing assay.

As further illustrated in FIG. 4, the methylation-genotype-imputation system 106 performs an act 406 of reducing values of genotype-likelihood metrics for the subset of candidate variant calls. To approximately account for errors introduced by methylation-sequencing-assay conversions, the methylation-genotype-imputation system 106 may reduce values of a subset of genotype-likelihood metrics for the subset of candidate variant calls within the variant call file. In particular, the methylation-genotype-imputation system 106 identifies a subset of candidate variant calls where nucleobase calls 416 diverge from identified bases in a reference genome 418. To illustrate, in some embodiments, candidate variant calls include thymine nucleobase calls corresponding to cytosine bases in the reference genome 418 and adenine nucleobase calls corresponding to guanine bases in the reference genome 418. In the alternative to thymine nucleobase calls, in certain cases, candidate variant calls include uracil nucleobase calls corresponding to cytosine bases in the reference genome 418.

FIG. 4 illustrates genotype-likelihood metrics 420 generated by a variant call model. In some cases, the genotype-likelihood metrics 420 comprise PHRED-scaled-genotype-likelihood metrics that have been normalized. In other implementations, the genotype-likelihood metrics 420 comprise other genotype-likelihood metrics, such as non-normalized genotype-likelihood metrics.

As indicated above, in some embodiments, the methylation-genotype-imputation system 106 reduces the values for the genotype-likelihood metrics 420. For instance, the methylation-genotype-imputation system 106 reduces levels of confidence for variant calls influenced by methylation sequencing assay conversions. In some implementations, the methylation-genotype-imputation system 106 modifies the genotype-likelihood metrics 420 to generate a reduced genotype-likelihood 430. In some examples, the methylation-genotype-imputation system 106 reduces the genotype-likelihood metrics 420 by a predetermined percentage value. For example, and as illustrated in FIG. 4, the methylation-genotype-imputation system 106 reduces a subset of genotype-likelihood metrics by 80%. In other embodiments, the methylation-genotype-imputation system 106 reduces a subset of genotype-likelihood metrics by another percentage, such as 70%, 75%, 85%, or any percentage.

As illustrated, for predicted cytosine-to-thymine conversions, the methylation-genotype-imputation system 106 modifies the value 0.97 of the genotype-likelihood metric by 80% to equal 0.194. In other implementations, the methylation-genotype-imputation system 106 dynamically determines the value by which to reduce the genotype-likelihood metrics. For example, the methylation-genotype-imputation system 106 may reduce values for the genotype-likelihood metrics 420 based on the methylation sequencing assay used. To illustrate, the methylation-genotype-imputation system 106 may reduce the genotype-likelihood metrics 420 for cytosine-to-thymine and guanine-to-adenine conversions but not for cytosine-to-uracil conversions based on determining that the methylation sequencing assay used does not convert cytosine bases to uracil bases. Likewise, in some embodiments, the methylation-genotype-imputation system 106 does not reduce the value of genotype-likelihood metrics for variants that do not correspond to enzymatic conversions by a given methylation sequencing assay, such as T>C, A>G, G>T, A>C, or C>A variant calls.

In some implementations, by contrast, the methylation-genotype-imputation system 106 modifies values of genotype-likelihood metrics by inflating or increasing the genotype-likelihood metrics. For example, at some genomic sites, the methylation-genotype-imputation system 106 may determine that imputation has a tendency to change correct genotype calls to incorrect genotype calls. To illustrate, the methylation-genotype-imputation system 106 may determine that a genotype imputation model tends to change a genotype call from a correct to an incorrect genotype call at a particular genomic coordinate or at a particular position a threshold number of nucleobases from (or within) a particular variant call (e.g., a C>T variant or a G>A variant call). Based on identifying a pattern of inaccuracy for a particular genomic site, the methylation-genotype-imputation system 106 can increase genotype-likelihood metrics for that site (e.g., by increasing a value of a genotype-likelihood metric by a particular percentage or ratio).

As suggested above, in some embodiments, the methylation-genotype-imputation system 106 applies a genotype imputation model, such as a hidden Markov model (HMM)-based genotype imputation model to nucleotide reads corresponding to a genomic region of a genomic sample. By applying a genotype imputation model, the methylation-genotype-imputation system 106 can determine posterior genotype likelihoods and haplotype calls for the genomic region. In accordance with one or more embodiments, FIG. 5 illustrates the methylation-genotype-imputation system 106 applying GLIMPSE as a genotype imputation model to determine posterior genotype likelihoods for a genomic region of a genomic sample.

Through imputation, the methylation-genotype-imputation system 106 imputes one or more genotype calls for a target genomic sample. In some embodiments, for a genomic coordinate of the target genomic sample, the methylation-genotype-imputation system 106 can determine a different genotype call from an initial genotype call determined by a variant call model. To illustrate, the methylation-genotype-imputation system 106 may impute a homozygous reference genotype call instead of a heterozygous variant genotype call or a homozygous variant genotype call initially determined by a variant call model. The methylation-genotype-imputation system 106 may also impute a heterozygous variant genotype call instead of a homozygous reference genotype call or a homozygous variant genotype call initially determined by the variant call model. In another example, the methylation-genotype-imputation system 106 imputes a homozygous variant genotype call instead of a heterozygous variant genotype call or the homozygous reference genotype call initially determined by the variant call model.

As shown in FIG. 5, for instance, the methylation-genotype-imputation system 106 determines reduced prior genotype likelihoods 504 and/or genotype likelihoods for a genomic region 500 from a genomic sample (e.g., a reference allele or alternate allele). More specifically, the methylation-genotype-imputation system 106 utilizes the reduced prior genotype likelihoods 504 corresponding to a subset of candidate variant calls exhibiting converted nucleobases from a methylation sequencing assay. For remaining variant and/or genotype calls that the methylation-genotype-imputation system 106 has determined are unaffected by methylation sequencing assay conversions, in some embodiments, the methylation-genotype-imputation system 106 utilizes initial (and unreduced) prior genotype likelihoods. For either the reduced prior genotype likelihoods 504 or the unreduced prior genotype likelihoods, in some embodiments, the methylation-genotype-imputation system 106 inputs PHRED-scaled-genotype-likelihood metrics as part of a VCF into the genotype imputation model.

In at least one example, a genotype call imputed by GLIMPSE is different from a genotype call generated by a variant call model (e.g., a combination model of DRAGEN VC and EpiDiverse) for any variant, not just cytosine-to-thymine and guanine-to-adenine conversions. For instance, the methylation-genotype-imputation system 106 determines a genotype call based on a highest posterior genotype likelihood output by the genotype imputation model (e.g., GLIMPSE) rather than an initial genotype call based on a highest prior genotype likelihood output by a variant call model (e.g., DRAGEN VC). Such a change in genotype call is more likely when the methylation-genotype-imputation system 106 reduces a prior genotype-likelihood metric for a candidate variant call exhibiting a converted nucleobase from a methylation sequencing assay (e.g., C>T or G>A variant calls). Accordingly, the imputed genotype call is the genotype corresponding to the highest posterior genotype likelihood.

As indicated by nucleotide reads 502, in some cases, the genomic region 500 exhibits low coverage (e.g., ≤8X read coverage). In some embodiments, the methylation-genotype-imputation system 106 uses a probabilistic variant call model (e.g., variant caller from DRAGEN) to determine the reduced prior genotype likelihoods 504 based on the nucleotide reads 502 from the genomic sample and an identified subset of genotype-likelihood metrics.

As further indicated by FIG. 5, the genomic region 500 corresponds to variable positions (or variable genomic coordinates) of a haplotype reference panel 506. In certain cases, the methylation-genotype-imputation system 106 further deconvolves a vector of the reduced prior genotype likelihoods 504 to two independent vectors of haplotype allele likelihoods (or, simply, haplotype likelihoods), where each vector corresponds to one of two complementary haplotypes.

Based on the haplotype likelihoods from the independent vectors, in some implementations, the methylation-genotype-imputation system 106 imputes two target haplotypes as haplotype calls using a haploid version of an HMM in an iterative process. As shown in FIG. 5, for instance, the methylation-genotype-imputation system 106 selects haplotypes 510 based on the haplotype reference panel 506 and target haplotypes 508 estimated for each genomic sample. After selecting haplotypes for a given genomic sample, the methylation-genotype-imputation system 106 stores reference and target versions of the selected haplotypes as a Positional Burrows Wheeler Transform (PBWT) 512.

As further shown in FIG. 5, in some embodiments, methylation-genotype-imputation system 106 samples haplotypes 514 in the PBWT 512 format by performing a linear-time-sampling algorithm based on a haplotype imputation version of HMM developed by Na Li and Matthew Stephens, “Modeling Linkage Disequilibrium and Identifying Recombination Hotspots Using Single-Nucleotide Polymorphism Data,” 165 Genetics 2213-2233 (2003), which is hereby incorporated by reference in its entirety. By performing the linear-time-sampling algorithm as part of sampler iterations, the methylation-genotype-imputation system 106 further determines (and updates) the phase of two imputed haplotypes for the genomic region 500 for a particular genomic sample.

Based on the imputed and phased haplotypes, as further shown in FIG. 5, the methylation-genotype-imputation system 106 determines posterior genotype likelihoods 516 that the genomic region 500 of the genomic sample exhibits particular genotypes (e.g., a reference allele or alternate allele). The methylation-genotype-imputation system 106 further determines haplotype calls 518 for the genomic region for each of the genomic sample. As indicated above, in some embodiments, the methylation-genotype-imputation system 106 uses a modified version of GLIMPSE developed by Rubinacci as a genotype imputation model.

As previously mentioned, the methylation-genotype-imputation system 106 improves the accuracy of variant calling relative to existing sequencing systems using methylation sequencing data. More specifically, in comparison with state-of-the-art systems that generate genotype calls with up to 0.95 precision and recall, the methylation-genotype-imputation system 106 provides best-in-class performance in both recall and precision. For example, the methylation-genotype-imputation system 106 may achieve 0.97 recall and 0.995 precision. In some implementations, the methylation-genotype-imputation system 106 pairs various methylation assay callers with variant callers to achieve different levels of accuracy. In accordance with one or more embodiments, FIGS. 6A and 6B illustrate performance results of various combinations of different methylation sequencing assay protocols and variant callers. By way of overview, FIGS. 6A-6B illustrate graphs indicating variant calling precision (e.g., “Single Nucleotide Polymorphism (SNP) Precision”) and variant calling recall (e.g., “SNP Recall”) when utilizing the following methylation sequencing assay protocols: whole genome sequencing (WGS), TAPS, BS, and EM. FIGS. 6A-6B also illustrate the impact of specific variant callers (e.g., DRAGEN VC, Epidiverse, BisSNP, Biscuit, CGmap, and Methylextract) on the variant calling precision and variant calling recall. The variant calling precision and the variant calling recall are determined based on a ground truth sample.

FIGS. 6A and 6B show the impact of various variant callers. DRAGEN VC comprises a bio-IT platform that provides secondary analysis of sequencing data. DRAGEN VC is described in additional detail in Illumina's technical note titled “DRAGEN Bio-IT Platform: Accurate, comprehensive, and efficient secondary analysis for NGS data” (available at https://www.illumina.com/content/dam/illumina/gcs/assembled-assets/marketing-literature/dragen-bio-it-data-sheet-m-gl-00680/dragen-bio-it-data-sheet-m-gl-00680.pdf), which is incorporated by reference as if fully set forth herein. Another variant caller, Epidiverse, is described in additional detail in Nunn, A, et al. “EpiDiverse Toolkit: a pipeline suite for the analysis of bisulfite sequencing data in ecological plant epigenetics,” NAR Genomics and Bioinformatics 3.4 (2021): Iqab106, which is incorporated by reference as if fully set forth herein. BisSNP is another variant caller described in greater detail by Liu, Y., Siegmund, K. D., Laird, P. W. et al. Bis-SNP: Combined DNA methylation and SNP calling for Bisulfite-seq data, Genome Biol 13, R61 (2012). https://doi.org/10.1186/gb-201201307-r61, which is incorporated by reference as if fully set forth herein. Additional information regarding Biscuit is found in Zhou W. BISCUIT: BISulfite-seq CUI Toolkit; 2020 (available at https://github.com/huishenlab/biscuit), which is incorporated by reference as if fully set forth herein. CGmap is described in greater detail in Weilong Guo, et al. CGmapTools improves the precision of heterozygous SNV calls and supports allele-specific methylation detection and visualization in bisulfite-sequencing data, Bioinformactics, Volume 34, Issue 3, 1 Feb. 2018, Pages 381-387, https://doi.org/10.1093/bioinformatics/btx595, which is incorporated by reference as if fully set forth herein. The methylextract variant caller is described in additional detail in Barturen G., et al. “MethylExtract: High-Quality methylation maps and SNV calling from whole genome bisulfite sequencing data,” F1000Research vol. 2 217. 15 Oct. 2013, doi: 10.12688/f1000research.2-217.v2, which is incorporated by reference as if fully set forth herein.

Generally, FIG. 6A illustrates graphs indicating variant calling precision (e.g., SNP Precision) and variant calling recall (e.g., SNP recall) resulting from different variant callers interacting with WGS and TAPS protocols. More specifically, FIG. 6A includes a graph 602 corresponding to a WGS protocol and a graph 604 corresponding to a TAPS protocol. The graph 602 comprises a graph portion 606 indicating that WGS paired with DRAGEN VC yields both a higher variant calling precision and a high variant calling recall relative to other variant callers. The graph 604 includes a graph portion 608 indicating that, when paired with the TAPS protocol, the Epidiverse variant caller also yields a higher variant precision and a higher variant recall relative to other variant callers.

Relatedly, FIG. 6B illustrates graphs indicating variant calling precision and variant calling recall resulting from the same variant callers depicted in FIG. 6A interacting with different methylation sequencing assay protocols than those depicted in FIG. 6A—that is, BS and EM protocols. Graph 610 shows variant calling precision and variant calling recall for various variant callers based on a BS protocol. As shown by graph portion 614 of the graph 610, none of the variant callers yield both variant calling precision and variant calling recall comparable to the same variant callers in combination with WGS or TAPS. In contrast, and as shown by graph portion 616 of graph 612, Epidiverse and BisSNP both have relatively higher variant calling precision and variant calling recall when paired with an EM protocol in comparison to a pairing with the BS protocol.

In at least one example, the methylation-genotype-imputation system 106 utilizes a whole genome sequencing (WGS) protocol in combination with the DRAGEN Variant Caller (VC). The methylation-genotype-imputation system 106 further applies GLIMPSE as a genotype imputation model to determine posterior genotype likelihoods. In some implementations, GLIMPSE generates a VCF or other base-call-output file. In at least one example, the methylation-genotype-imputation system 106 does not report the reduced genotype-likelihood in the VCF. The methylation-genotype-imputation system 106 assigns a new format field with genotype probabilities (GPs) from GLIMPSE to all variants in the reference panel. Furthermore, the methylation-genotype-imputation system 106 may update target genotype (GT) and the PHRED-scaled quality score for the assertion made in ALT (QUAL) metrics to show the imputed genotype and the QUAL score calculated from the GPs.

In some cases, the methylation-genotype-imputation system 106 may utilize two or more variant callers in combination to further improve accuracy of variant calls. In one example, the methylation-genotype-imputation system 106 may modify the output of a first variant caller and utilize a second variant caller to analyze the modified output. Furthermore, the methylation-genotype-imputation system 106 may modify one or more of the variant callers so that they can work in conjunction. To illustrate, in some embodiments, the methylation-genotype-imputation system 106 modifies and combines DRAGEN VC and EpiDiverse to boost variant calling performance. Generally, EpiDiverse effectively masks some base calls (e.g., C-to-T conversions) and generates a Binary Alignment Map (BAM) file. In some cases, the methylation-genotype-imputation system 106 modifies DRAGEN VC to accept the EpiDiverse BAM file as input. More specifically, the methylation-genotype-imputation system 106 modifies code for DRAGEN VC to allow disabling of N base interpretation during variant calling.

Typically, N base interpretation reduces noise and designates some nucleobase calls as “no calls” because the quality score (or some other sequencing metric) is too low to pass filter. N base calls are either present in nucleotide reads at the base calling stage or assigned within DRAGEN VC when base quality is below a certain threshold. The low base qualities assigned by EpiDiverse to converted nucleobases lead to T-to-N base conversions. In some examples, the T-to-N base conversion reduces the quality of DRAGEN VC variant calling. Thus, in some implementations, the methylation-genotype-imputation system 106 modifies DRAGEN VC to receive BAM files as input and disable N-base interpretation.

The combination of EpiDiverse and DRAGEN VC yields improvements to both precision and recall relative to single SNP callers illustrated in FIGS. 6A-6B. For example, and as shown in FIGS. 6A-6B, DRAGEN VC has low precision on its own for TAPS protocol, BS protocol, and EM protocol. More specifically, DRAGEN VC on its own yields a higher number of false positive calls. This is due, in part, to methylation conversions that are considered as heterozygous SNPs. EpiDiverse, on its own performs similarly to DRAGEN VC. EpiDiverse calls SNPs with greater precision than DRAGEN VC when TAPS, BS, or EM protocols are used. However, EpiDiverse SNP calls suffer from both lower recall and lower precision using BS protocol. The combination of EpiDiverse and DRAGEN VC boosts variant calling performance to 0.99 recall and 0.995 precision. The performance of SNP calling (both precision and recall) by a combination of DRAGEN VC and EpiDiverse is boosted even more by utilizing a genotype imputation model (e.g., GLIMPSE).

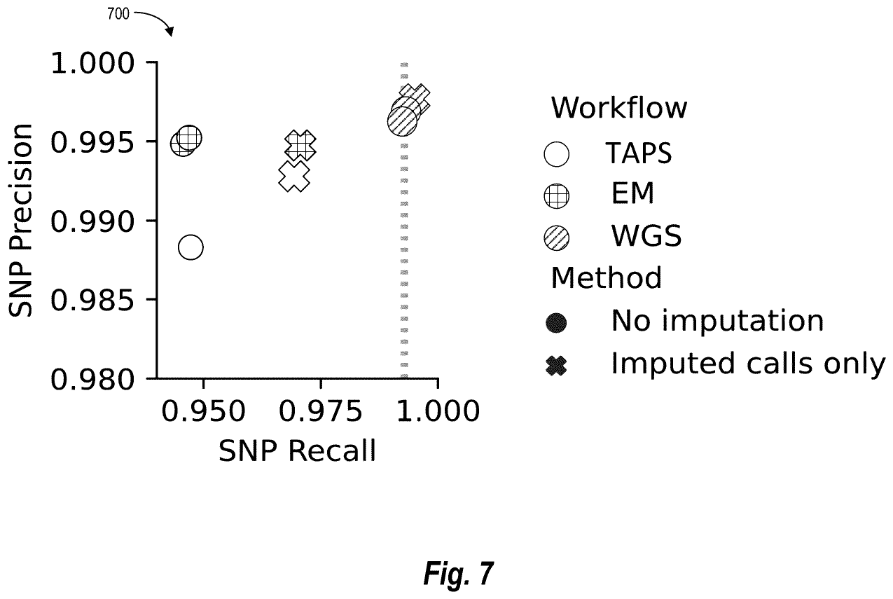

As previously mentioned, the methylation-genotype-imputation system 106 utilizes imputation to further improve the accuracy of variant calls. In accordance with one or more embodiments, FIG. 7 illustrates a graph 700 demonstrating how imputation boosts variant calling performance. The graph 700 shows variant calling precision and variant calling recall for TAPS, EM, and WGS with and without imputation. As shown, imputation positively affects both TAPS and EM. More specifically, both TAPS and EM have improved recall. The variant calling precision for TAPS and EM remain stable. Furthermore, even though WGS without imputation has relatively good performance, imputation provides additional improvements to both precision and recall.

As further shown in FIG. 7, the graph 700 also includes a recall limit represented by a dashed line. The recall limit comprises a function of the number of samples in the reference panel or the size of the reference panel. In some examples, the size of the reference panel is theoretically limited by the number of variants that the methylation-genotype-imputation system 106 may recover because some of the variants in truth sets are still missing from that panel. One way of increasing the recall limit is by increasing the size of the reference panel. More specifically, sequencing more individuals within the reference panel increases access to more variants in the ground truth of a particular individual.

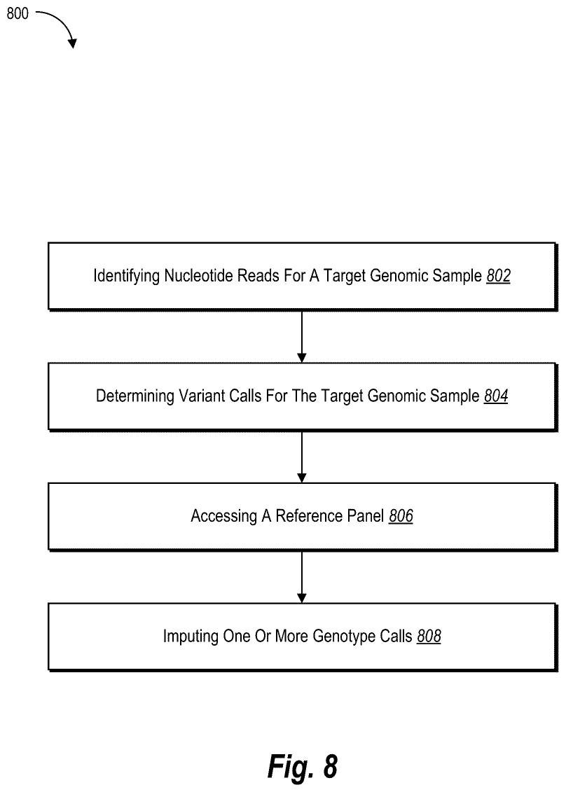

FIGS. 1-7, the corresponding text, and the examples provide a number of different methods, systems, devices, and non-transitory computer-readable media of the methylation-genotype-imputation system 106. In addition to the foregoing, one or more implementations can also be described in terms of flowcharts comprising acts for accomplishing a particular result, as shown in FIG. 8. FIG. 8 illustrates a flowchart of a series of acts 800 of imputing one or more genotype calls in accordance with one or more embodiments of the present disclosure. While FIG. 8 illustrates acts according to one embodiment, alternative embodiments may omit, add to, reorder, and/or modify any of the acts shown in FIG. 8. The acts of FIG. 8 can be performed as part of a method. Alternatively, a non-transitory computer readable storage medium can comprise instructions that, when executed by one or more processors, cause a computing device or a system to perform the acts depicted in FIG. 8. In still further embodiments, a system comprising at least one processor and a non-transitory computer readable medium comprising instructions that, when executed by one or more processors, cause the system to perform the acts of FIG. 8.

As shown in FIG. 8, the series of acts 800 includes an act 802 of identifying nucleotide reads for a target genomic sample. In particular, the act 802 comprises identifying, for a target genomic sample, nucleotide reads comprising one or more nucleobases converted by a methylation sequencing assay. In some implementations, identifying nucleotide reads comprising one or more nucleobases converted by the methylation sequencing assay comprises identifying the nucleotide reads comprising thymine bases or uracil bases converted from cytosine bases by the methylation sequencing assay.

The series of acts 800 includes an act 804 of determining variant calls for the target genomic sample. In particular, the act 804 comprises determining variant calls for the target genomic sample based on an alignment of the nucleotide reads with a reference genome.

FIG. 8 further illustrates an act 806 of accessing a reference panel. In particular, the act 806 comprises accessing a reference panel comprising marker variants for different haplotypes corresponding to a target genomic region of the target genomic sample.

The series of acts 800 further includes the act 808 of imputing one or more genotype calls. In particular, the act 808 comprises imputing one or more genotype calls for the target genomic sample based on a comparison of a subset of variant calls for the target genomic sample and the marker variants from the reference panel. In some embodiments, imputing the one or more genotype calls for the target genomic sample comprises imputing, for a genomic coordinate of the target genomic sample, a genotype call differing from an initial variant call of the variant calls determined by a variant call model. Furthermore, in some implementations, imputing the one or more genotype calls for the target genomic sample comprises imputing, for a genomic coordinate of the target genomic sample, a genotype call differing from an initial genotype call determined by a variant call model by: imputing a homozygous reference genotype call instead of a heterozygous variant genotype call or a homozygous variant genotype call initially determined by the variant call model, imputing the heterozygous variant genotype call instead of the homozygous reference genotype call or the homozygous variant genotype call initially determined by the variant call model, and imputing the homozygous variant genotype call instead of the heterozygous variant genotype call or the homozygous reference genotype call initially determined by the variant call model. Furthermore, in some implementations, imputing the one or more genotype calls for the target genomic sample comprises imputing a genotype call for a single nucleotide polymorphism (SNP), a deletion, an insertion, a duplication, an inversion, a translocation, or a copy number variation (CNV).

In some embodiments, the series of acts 800 includes additional acts of generating a variant call file comprising the variant calls for the target genomic sample and reducing values of a subset of genotype-likelihood metrics for a subset of candidate variant calls within the variant call file to approximately account for errors introduced by the methylation sequencing assay. In some implementations, reducing the values of the subset of genotype-likelihood metrics for the subset of candidate variant calls comprises: reducing values of PHRED-scaled-genotype-likelihood metrics of thymine-base calls at genomic coordinates for which the reference genome comprises cytosine bases and reducing values of PHRED-scaled-genotype-likelihood metrics of adenine-base calls at genomic coordinates for which the reference genome comprises guanine bases.

In some cases, the series of acts 800 further comprises determining that detected thymine bases from the nucleotide reads differ from reference cytosine bases within the reference genome, wherein the detected thymine bases comprise uracil bases that have been converted from cytosine bases by the methylation sequencing assay and subsequently detected as thymine bases by a sequencing device instead of detected as the uracil bases and generating methylation-level values indicating levels of methylation of the cytosine bases within the target genomic sample.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the methylation-genotype-imputation system 106 can include software, hardware, or both. For example, the components of the methylation-genotype-imputation system 106 can include one or more instructions stored on a computer-readable storage medium and executable by processors of one or more computing devices (e.g., the user client device 110). When executed by the one or more processors, the computer-executable instructions of the methylation-genotype-imputation system 106 can cause the computing devices to perform the bubble detection methods described herein. Alternatively, the components of the methylation-genotype-imputation system 106 can comprise hardware, such as special purpose processing devices to perform a certain function or group of functions. Additionally, or alternatively, the components of the methylation-genotype-imputation system 106 can include a combination of computer-executable instructions and hardware.

Furthermore, the components of the methylation-genotype-imputation system 106 performing the functions described herein with respect to the methylation-genotype-imputation system 106 may, for example, be implemented as part of a stand-alone application, as a module of an application, as a plug-in for applications, as a library function or functions that may be called by other applications, and/or as a cloud-computing model. Thus, components of the methylation-genotype-imputation system 106 may be implemented as part of a stand-alone application on a personal computing device or a mobile device. Additionally, or alternatively, the components of the methylation-genotype-imputation system 106 may be implemented in any application that provides sequencing services including, but not limited to Illumina BaseSpace, BeadArray, BeadChip, Illumina DRAGEN, Infinium Methylation Assay, or Illumina TruSight software. “Illumina,” “BeadArray,” “BeadChip,” “BaseSpace,” “DRAGEN,” “Infinium Methylation Assay,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCS, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 9 illustrates a block diagram of a computing device 900 that may be configured to perform one or more of the processes described above. One will appreciate that one or more computing devices such as the computing device 900 may implement the methylation-genotype-imputation system 106 and the sequencing system 104. As shown by FIG. 9, the computing device 900 can comprise a processor 902, a memory 904, a storage device 906, an I/O interface 908, and a communication interface 910, which may be communicatively coupled by way of a communication infrastructure 912. In certain embodiments, the computing device 900 can include fewer or more components than those shown in FIG. 9. The following paragraphs describe components of the computing device 900 shown in FIG. 9 in additional detail.

In one or more embodiments, the processor 902 includes hardware for executing instructions, such as those making up a computer program. As an example, and not by way of limitation, to execute instructions for dynamically modifying workflows, the processor 902 may retrieve (or fetch) the instructions from an internal register, an internal cache, the memory 904, or the storage device 906 and decode and execute them. The memory 904 may be a volatile or non-volatile memory used for storing data, metadata, and programs for execution by the processor(s). The storage device 906 includes storage, such as a hard disk, flash disk drive, or other digital storage device, for storing data or instructions for performing the methods described herein.

The I/O interface 908 allows a user to provide input to, receive output from, and otherwise transfer data to and receive data from computing device 900. The I/O interface 908 may include a mouse, a keypad or a keyboard, a touch screen, a camera, an optical scanner, network interface, modem, other known I/O devices or a combination of such I/O interfaces. The I/O interface 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 908 is configured to provide graphical data to a display for presentation to a user. The graphical data may be representative of one or more graphical user interfaces and/or any other graphical content as may serve a particular implementation.

The communication interface 910 can include hardware, software, or both. In any event, the communication interface 910 can provide one or more interfaces for communication (such as, for example, packet-based communication) between the computing device 900 and one or more other computing devices or networks. As an example, and not by way of limitation, the communication interface 910 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 910 may facilitate communications with various types of wired or wireless networks. The communication interface 910 may also facilitate communications using various communication protocols. The communication infrastructure 912 may also include hardware, software, or both that couples components of the computing device 900 to each other. For example, the communication interface 910 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A method comprising:
identifying, for a target genomic sample, nucleotide reads comprising one or more nucleobases converted by a methylation sequencing assay;
determining variant calls for the target genomic sample based on an alignment of the nucleotide reads with a reference genome;
accessing a reference panel comprising marker variants for different haplotypes corresponding to a target genomic region of the target genomic sample; and
imputing one or more genotype calls for the target genomic sample based on a comparison of a subset of variant calls for the target genomic sample and the marker variants from the reference panel.

2. The method of claim 1, wherein identifying nucleotide reads comprising one or more nucleobases converted by the methylation sequencing assay comprises identifying the nucleotide reads comprising thymine bases or uracil bases converted from cytosine bases by the methylation sequencing assay.

3. The method of claim 1, further comprising:
generating a variant call file comprising the variant calls for the target genomic sample; and
reducing values of a subset of genotype-likelihood metrics for a subset of candidate variant calls within the variant call file to approximately account for errors introduced by the methylation sequencing assay.

4. The method of claim 3, wherein reducing the values of the subset of genotype-likelihood metrics for the subset of candidate variant calls comprises:
reducing values of PHRED-scaled-genotype-likelihood metrics of thymine-base calls at genomic coordinates for which the reference genome comprises cytosine bases; and
reducing values of PHRED-scaled-genotype-likelihood metrics of adenine-base calls at genomic coordinates for which the reference genome comprises guanine bases.

5. The method of claim 1, further comprising:
determining that detected thymine bases from the nucleotide reads differ from reference cytosine bases within the reference genome, wherein the detected thymine bases comprise uracil bases that have been converted from cytosine bases by the methylation sequencing assay and subsequently detected as thymine bases by a sequencing device instead of detected as the uracil bases; and
generating methylation-level values indicating levels of methylation of the cytosine bases within the target genomic sample.

6. The method of claim 1, wherein imputing the one or more genotype calls for the target genomic sample comprises imputing, for a genomic coordinate of the target genomic sample, a genotype call differing from an initial variant call of the variant calls determined by a variant call model.

7. The method of claim 1, wherein imputing the one or more genotype calls for the target genomic sample comprises imputing, for a genomic coordinate of the target genomic sample, a genotype call differing from an initial genotype call determined by a variant call model by:
imputing a homozygous reference genotype call instead of a heterozygous variant genotype call or a homozygous variant genotype call initially determined by the variant call model;
imputing the heterozygous variant genotype call instead of the homozygous reference genotype call or the homozygous variant genotype call initially determined by the variant call model; or
imputing the homozygous variant genotype call instead of the heterozygous variant genotype call or the homozygous reference genotype call initially determined by the variant call model.

8. The method of claim 1, wherein imputing the one or more genotype calls for the target genomic sample comprises imputing a genotype call for a single nucleotide polymorphism (SNP), a deletion, an insertion, a duplication, an inversion, a translocation, or a copy number variation (CNV).

9. A system comprising:
at least one processor; and
a non-transitory computer-readable medium comprising instructions that, when executed by the at least one processor, cause the system to:
identify, for a target genomic sample, nucleotide reads comprising one or more nucleobases converted by a methylation sequencing assay;
determine variant calls for the target genomic sample based on an alignment of the nucleotide reads with a reference genome;
access a reference panel comprising marker variants for different haplotypes corresponding to a target genomic region of the target genomic sample; and
impute one or more genotype calls for the target genomic sample based on a comparison of a subset of variant calls for the target genomic sample and the marker variants from the reference panel.

10. The system of claim 9, further comprising instructions that, when executed by the at least one processor, cause the system to identify nucleotide reads comprising one or more nucleobases converted by the methylation sequencing assay by identifying the nucleotide reads comprising thymine bases or uracil bases converted from cytosine bases by the methylation sequencing assay.

11. The system of claim 9, further comprising instructions that, when executed by the at least one processor, cause the system to:
generate a variant call file comprising the variant calls for the target genomic sample; and
reduce values of a subset of genotype-likelihood metrics for a subset of candidate variant calls within the variant call file to approximately account for errors introduced by the methylation sequencing assay.

12. The system of claim 11, further comprising instructions that, when executed by the at least one processor, cause the system to reduce the values of the subset of genotype-likelihood metrics for the subset of candidate variant calls by:
reducing values of PHRED-scaled-genotype-likelihood metrics of thymine-base calls at genomic coordinates for which the reference genome comprises cytosine bases; and
reducing values of PHRED-scaled-genotype-likelihood metrics of adenine-base calls at genomic coordinates for which the reference genome comprises guanine bases.

13. The system of claim 9, further comprising instructions that, when executed by the at least one processor, cause the system to:
determine that detected thymine bases from the nucleotide reads differ from reference cytosine bases within the reference genome, wherein the detected thymine bases comprise uracil bases that have been converted from cytosine bases by the methylation sequencing assay and subsequently detected as thymine bases by a sequencing device instead of detected as the uracil bases; and
generate methylation-level values indicating levels of methylation of the cytosine bases within the target genomic sample.

14. The system of claim 9, further comprising instructions that, when executed by the at least one processor, cause the system to impute the one or more genotype calls for the target genomic sample by imputing, for a genomic coordinate of the target genomic sample, a genotype call differing from an initial variant call of the variant calls determined by a variant call model.

15. The system of claim 9, further comprising instructions that, when executed by the at least one processor, cause the system to impute the one or more genotype calls for the target genomic sample by imputing, for a genomic coordinate of the target genomic sample, a genotype call differing from an initial genotype call determined by a variant call model by:
imputing a homozygous reference genotype call instead of a heterozygous variant genotype call or a homozygous variant genotype call initially determined by the variant call model;
imputing the heterozygous variant genotype call instead of the homozygous reference genotype call or the homozygous variant genotype call initially determined by the variant call model; or
imputing the homozygous variant genotype call instead of the heterozygous variant genotype call or the homozygous reference genotype call initially determined by the variant call model.

16. A non-transitory computer-readable medium storing instructions that, when executed by at least one processor, cause a computing device to:
identify, for a target genomic sample, nucleotide reads comprising one or more nucleobases converted by a methylation sequencing assay;
determine variant calls for the target genomic sample based on an alignment of the nucleotide reads with a reference genome;
access a reference panel comprising marker variants for different haplotypes corresponding to a target genomic region of the target genomic sample; and
impute one or more genotype calls for the target genomic sample based on a comparison of a subset of variant calls for the target genomic sample and the marker variants from the reference panel.

17. The non-transitory computer-readable medium of claim 16, further comprising instructions that, when executed by the at least one processor, cause the computing device to impute the one or more genotype calls for the target genomic sample by imputing, for a genomic coordinate of the target genomic sample, a genotype call differing from an initial genotype call determined by a variant call model by:
imputing a homozygous reference genotype call instead of a heterozygous variant genotype call or a homozygous variant genotype call initially determined by the variant call model;
imputing the heterozygous variant genotype call instead of the homozygous reference genotype call or the homozygous variant genotype call initially determined by the variant call model; or
imputing the homozygous variant genotype call instead of the heterozygous variant genotype call or the homozygous reference genotype call initially determined by the variant call model.

18. The non-transitory computer-readable medium of claim 17, further comprising instructions that, when executed by the at least one processor, cause the computing device to impute the one or more genotype calls for the target genomic sample comprises imputing a genotype call for a single nucleotide polymorphism (SNP), a deletion, an insertion, a duplication, an inversion, a translocation, or a copy number variation (CNV).

19. The non-transitory computer-readable medium of claim 17, further comprising instructions that, when executed by the at least one processor, cause the computing device to identify nucleotide reads comprising one or more nucleobases converted by the methylation sequencing assay by identifying the nucleotide reads comprising thymine bases or uracil bases converted from cytosine bases by the methylation sequencing assay.

20. The non-transitory computer-readable medium of claim 17, further comprising instructions that, when executed by the at least one processor, cause the computing device to:
generate a variant call file comprising the variant calls for the target genomic sample; and
reduce values of a subset of genotype-likelihood metrics for a subset of candidate variant calls within the variant call file to approximately account for errors introduced by the methylation sequencing assay.
